(19) United States
(12) Patent Application Publication
(10) Pub. No.: US 2026/0115373 A1
(43) Pub. Date: Apr. 30, 2026

(54) CONNECTING UNIT FOR VACUUM-TIGHT FLUIDIC CONNECTION OF A VACUUM WOUND DRESSING TO A VACUUM SOURCE, VACUUM WOUND TREATMENT KIT AND VACUUM WOUND TREATMENT SYSTEM

(71) Applicant: PAUL HARTMANN AG, Heidenheim (DE)

(72) Inventor: Martin DEIBLER, Herbrechtingen (DE)

(73) Assignee: PAUL HARTMANN AG, Heidenheim (DE)

(21) Appl. No.: 19/140,258

(22) PCT Filed: Dec. 6, 2023
(86) PCT No.: PCT/EP2023/084531

§ 371 Date: Jun. 17, 2025

(30) Foreign Application Priority Data
Dec. 19, 2022 (DE) 10 2022 133 931.9

(51) Int. Cl. A61M 1/00

(52) U.S. Cl. CPC A61M 1/913; A61M 1/784; A61M 2205/7536

(57) ABSTRACT

The invention relates to a connecting unit (20) for vacuum-tight fluidic connection of a vacuum wound dressing (16) to a vacuum source (12), comprising an elongated single- or multi-lumen connecting tube (22), having a distal end section (26) and proximal end section (24), and wherein the distal end section (26) can be fluidically connected to the vacuum wound dressing (16) in a vacuum-tight manner, and comprising a connection element (34) having a fluid inlet (36) and a fluid outlet, wherein an inlet section (38) of the connection element (34) including the fluid inlet (36) projects into a tube lumen (40) of the connecting tube (22) in the region of the proximal end section (24) , and wherein the fluid outlet is arranged outside of the tube lumen (40) and can be fluidically connected to the vacuum source (12) in a vacuum-tight manner; according to the invention, a filter unit (42) is arranged on the end section (38), which is associated with the fluid inlet (36) and is permeable to air and non-permeable to liquid.

The present invention relates to a connection unit for vacuum-tight fluidic connection of a vacuum wound dressing to a vacuum source. The present invention also relates to a vacuum wound treatment kit comprising such a connection unit. In addition, the present invention relates to a vacuum wound treatment system comprising a vacuum source and such a vacuum wound treatment kit.

The vacuum treatment of wounds, also referred to as NPWT=Negative-Pressure Wound Therapy, is an innovative method of treating wounds with a wide range of indications. These include, for example, acute skin and soft tissue defects, wound healing disorders, chronic wounds etc. The aim of vacuum treatment is to stimulate the growth of granulation tissue and to promote the healing process. In the context of vacuum treatment, this aim is pursued by producing a vacuum in the region of a wound, thereby enabling wound exudate to be drained effectively from the wound.

Vacuum wound dressings used in vacuum treatment typically comprise an air-impermeable covering layer for airtight sealing of the wound. A connection opening for vacuum-tight fluidic connection of the wound space to a vacuum source is formed in the covering layer. When the vacuum wound dressing is applied to a wound as intended and the vacuum source has been fluidically connected to the connection opening in a vacuum-tight manner, a vacuum can be produced in the wound space by the vacuum source. To fluidically connect the vacuum wound dressing to the vacuum source in a vacuum-tight manner, a connection unit comprising an elongate single-or multi-lumen connecting tube is typically used. A distal end section of the connecting tube can be fluidically connected to the vacuum wound dressing in a vacuum-tight manner. A proximal end section of the connecting tube can be fluidically connected to the vacuum source in a vacuum-tight manner.

A connection unit of the type in question is known from WO 2016 184 916 A1, for example. In order to produce the connection of the proximal end section of the connecting tube to the vacuum source, the connection unit comprises a connection element with a fluid inlet and a fluid outlet. An inlet section of the connection element, said inlet section comprising the fluid inlet, projects into a tube lumen of the connecting tube in the region of the proximal end section. In contrast, the fluid outlet of the connection element is arranged outside the tube lumen and can be fluidically connected to the vacuum source in a vacuum-tight manner. In the case of the connection unit disclosed in WO 2016 184 917 A1, a filter unit is arranged on the distal end section of the connecting tube. The filter unit prevents wound exudate from getting into the connecting tube and the downstream vacuum source when the connection unit is used as intended. Otherwise, the vacuum source could be contaminated and/or damaged by the wound exudate. On account of the arrangement of the filter unit on the distal end section or adjacent to the vacuum wound dressing, however, the filter unit may already be wetted with wound exudate and clogged before the absorption capacity of the vacuum wound dressing for wound exudate is exhausted. The vacuum wound dressing must then be changed at an unnecessarily early stage.

It is the underlying object of the invention to provide a connection unit which, when used as intended in vacuum treatment, effectively prevents wound exudate from entering the vacuum source. At the same time, the best possible use should be made of the absorption capacity of the vacuum wound dressing for wound exudate.

According to the invention, this object is achieved by a connection unit as claimed in claim 1, by a vacuum wound treatment kit as claimed in claim 18, and by a vacuum wound treatment system as claimed in claim 19.

The dependent claims and the description indicate advantageous variants and embodiments.

According to the invention, therefore, a connection unit for vacuum-tight fluidic connection of a vacuum wound dressing to a vacuum source is provided. The connection unit comprises an elongate single- or multi-lumen connecting tube. A distal end section of the connecting tube can be fluidically connected to the vacuum wound dressing in a vacuum-tight manner.

Here, a “vacuum-tight fluidic connection” should be understood to mean that, taking into account the vacuum source used, the vacuum necessary for the vacuum treatment of wounds can be maintained in the fluidically interconnected cavities. In the context of vacuum treatment, a pressure difference between the air pressure within the wound dressing and the ambient air pressure that is at least 20 mm Hg (millimeters of mercury) to a maximum of 250 mm Hg is typically set. 1 mm Hg corresponds to one torr or 133.322 Pa (pascals).

The terms “distal” and “proximal” describe the arrangement relative to the vacuum source. A proximal section of an element is situated closer to the vacuum source than a distal section of the same element. Thus, the proximal end section of the connecting tube is closer to the vacuum source than the distal end section, for example, when the connecting tube is used as intended.

The connection unit according to the invention furthermore comprises a connection element, which comprises a fluid inlet and a fluid outlet. An inlet section of the connection element, said inlet section comprising the fluid inlet, projects into a tube lumen of the connecting tube in the region of the proximal end section. The fluid outlet is arranged outside the tube lumen and can be fluidically connected to the vacuum source in a vacuum-tight manner.

Provision is now made for a filter unit to be arranged on the inlet section of the connection element, which filter unit is assigned to the fluid inlet and is of air-permeable and liquid-impermeable design. Owing to the assignment of the filter unit to the fluid inlet, fluid must pass through the filter unit in order to get from the tube lumen through the fluid inlet into the connection element. Because the filter unit is of air-permeable design, a vacuum can be communicated via the filter unit from the vacuum source to the tube lumen, the vacuum wound dressing and the wound space. Because the filter unit is of liquid-impermeable design, entry of wound exudate into the vacuum source is effectively avoided. As already mentioned, such an entry of wound exudate could lead to contamination of and/or damage to the vacuum source. The arrangement of the filter unit on the inlet section ensures that the absorption capacity of the vacuum wound dressing can be exploited in the best possible way. In particular, the filter unit is clogged by wound exudate only when the absorption capacity of the vacuum wound dressing has been exceeded and wound exudate consequently gets as far as the proximal end section of the connecting tube. The filter unit is preferably arranged on an outer contour of the inlet section. Such an embodiment of the connection unit is easy to implement in terms of manufacturing technology.

The connecting tube can be of single-lumen or multi-lumen design. In the case of a single-lumen connecting tube, there is only a single tube lumen, which extends from the distal end section to the proximal end section. When the connection unit is used as intended, the tube lumen serves as a suction lumen. In the case of a multi-lumen connecting tube, the tube lumen is divided into a number of partial lumens, which each extend from the distal end section to the proximal end section. The partial lumens are preferably connected in parallel with one another to an inlet opening formed in the distal end section, wherein each of the partial lumens is assigned a respectively dedicated passage in the region of the proximal end section. In the case of such a multi-lumen connecting tube, a first one of the partial lumens serves as the suction lumen when the connection unit is used as intended. The connection element projects through the assigned passage into this partial lumen in the region of the proximal end section. A second one of the partial lumens can be used as a purging lumen and/or as a measurement lumen, for example.

According to one preferred embodiment, it is envisaged that the filter unit comprises at least one air-permeable and liquid-impermeable membrane filter. A membrane filter can be obtained at a favorable cost and can be adapted precisely to the fluid inlet. The membrane filter can preferably be joined to the inlet section, in particular to the outer contour of the inlet section, by an adhesive joint or by a welded joint. As a particular preference, the filter unit comprises a membrane filter designed as a PTFE membrane filter. A membrane filter of this kind has an advantageous dewetting behavior. This means that the membrane filter is not permanently clogged by just slight wetting with wound exudate. Instead, the wound exudate flows back from the membrane filter, and therefore the membrane filter remains air-permeable.

The fluid inlet comprises at least one inlet opening. In particular, the fluid inlet comprises just one inlet opening. However, it is preferred that the fluid inlet comprises a plurality of inlet openings. As a particular preference, the plurality of inlet openings is covered by a common membrane filter of the filter unit. This has the advantage that the number of individual parts of the filter unit is small, this being associated with low production costs. Alternatively, the filter unit comprises a plurality of membrane filters, wherein the membrane filters each cover a different inlet opening or several cover respectively different inlet openings.

According to one preferred embodiment, it is envisaged that the inlet section is designed in such a way as to be widened transversely to the longitudinal extent of the connection element. The transverse extent of the inlet section is therefore greater than the transverse extent of a section of the connection element which directly adjoins the inlet section in the direction of the fluid outlet. This has the advantage that a fluid inlet with a large opening area can be obtained in the inlet section. Accordingly, it is also possible to enlarge the effective filter area of the filter unit. This reduces the probability that the filter unit will be clogged prematurely, that is to say before the absorption capacity of the wound dressing is exceeded.

As a preferred option, at least one inlet opening of the fluid inlet is formed in a flat wall section of the inlet section. Such an inlet opening can be covered in a particularly reliable manner by a membrane filter. As a preferred option, all the inlet openings of the fluid inlet are formed in a flat wall section of the inlet section or in a plurality of flat wall sections of the inlet section.

According to one preferred embodiment, it is envisaged that the inlet section comprises a first wall section and a second wall section, which come together at an acute angle at a front edge of the inlet section, and that the fluid inlet comprises at least one inlet opening formed in the first wall section and/or at least one inlet opening formed in the second wall section. Such a design of the inlet section on the one hand has the advantage that a single membrane filter can easily be mounted on the inlet section in such a way that it is arranged both on the first wall section and on the second wall section. For this purpose, the membrane filter is folded or bent around the front edge of the inlet section, for example. Moreover, the inlet section, which tapers to a point at the front edge, can easily be inserted between any layered elements, e.g. supporting layers, that may be present in the tube lumen. The layered elements then prevent the tube wall of the connecting tube resting flat against the first wall section and the second wall section and thereby closing the fluid inlet. The first wall section and the second wall section preferably come together at an acute angle, which is at most 60°, in particular at most 45°, in particular at most 30°. The first wall section and/or the second wall section are preferably of flat design. The front edge is preferably aligned orthogonally to the longitudinal extent of the connecting tube.

As a particular preference, at least one inlet opening is formed both in the first wall section and in the second wall section. It is thereby possible to obtain a fluid inlet with a particularly large opening area. As a particular preference, there is a membrane filter which is folded or bent around the front edge and covers both the at least one inlet opening in the first wall section and the at least one inlet opening in the second wall section. Alternatively, the at least one inlet opening in the first wall section and the at least one inlet opening in the second wall section are each covered by a different membrane filter.

According to one preferred embodiment, it is envisaged that the inlet section comprises a third wall section and a fourth wall section, which are extended orthogonally to the first wall section and the second wall section, and that the third wall section and the fourth wall section are of closed design. Covering an inlet opening in the third wall section or the fourth wall section would be possible only with increased effort. Accordingly, such an inlet opening is preferably dispensed with.

The inlet section is preferably of wedge-shaped design. A wedge-shaped inlet section combines the above-described preferred features in an advantageous manner.

According to one preferred embodiment, it is envisaged that the connecting tube comprises a first ply and a second ply, wherein the plies are materially joined to one another in their edge regions and jointly surround the tube lumen. The first ply and the second ply jointly form the tube wall of the connecting tube. The formation of the tube wall from the two plies makes the connection unit easier to assemble. The first ply and the second ply are preferably designed as film plies. Accordingly, the connecting tube is a film tube. The plies are preferably welded or adhesively bonded to one another in their edge regions.

According to one preferred embodiment, it is envisaged that the connection element projects through a passage into the tube lumen, which passage is formed between the edge region of the first ply and the edge region of the second ply. Such a connection unit can be obtained with little manufacturing effort. In the course of manufacture, the connection element is first of all arranged between the two plies, for example. Only then are the two plies materially joined to one another in their edge regions. In addition, there is the advantage that reliable sealing of the tube lumen can be achieved because there is a large contact area between the outer contour of the connection element and the edge regions of the plies. The edge regions of the two plies preferably rest fluid-tightly against the outer contour of the connection element. According to one alternative embodiment, the connection element projects through a passage formed in the first ply or in the second ply into the tube lumen.

According to one preferred embodiment, it is envisaged that a section of the connection element which is arranged in the passage comprises at least one protrusion for positive-locking and/or clamp-locking retention of the section in the passage. The connection element is thereby reliably prevented from sliding out of the proximal end section. The protrusion or protrusions are preferably formed integrally with the connection element.

A supporting unit is preferably arranged in the tube lumen, said unit supporting the connecting tube against collapse, in particular vacuum-induced collapse, and allowing fluid to pass through in the longitudinal extent of the tube lumen, wherein the supporting unit comprises at least one supporting layer, and wherein the connection element and the supporting layer overlap in some region or regions. Because the supporting layer and the connection element overlap in some region or regions, the tube wall is prevented from resting against that wall section of the inlet section which faces the supporting layer. Accordingly, an inlet opening formed in the relevant wall section is not closed by the tube wall.

According to one preferred embodiment, it is envisaged that the supporting layer is formed by a flat material web section which, in order to support the connecting tube, is formed in a structured manner by raised portions formed integrally with a surface of the flat material web section, wherein a continuous interspace, which allows fluid to pass through in the longitudinal extent of the tube lumen, is formed between the raised portions. A flat material web section designed as above can be obtained at low cost. This results, in particular, from the fact that the design of the raised portions can advantageously be integrated into the production of the flat material web section. A flat material web section is a section of a flat material web. For example, the flat material web section is cut out of a flat material web. The raised portions are preferably already formed in the flat material web. However, it is also possible for the raised portions to be formed only in the flat material web section. In particular, the flat material web is a plastic film web, such that the flat material web section is designed as a plastic film.

According to one preferred embodiment, it is envisaged that the supporting unit comprises at least two supporting layers arranged stacked one on top of the other, and that the connection element projects between the supporting layers. This is a particularly effective way of avoiding the tube wall coming into contact with the inlet section and thereby closing the fluid inlet.

According to one preferred embodiment, the connection element comprises a vacuum tube, which is formed integrally with the connection element and can be fluidically connected to the vacuum source in a vacuum-tight manner. It is thereby possible to reduce the number of individual parts required. In such an embodiment, the fluid outlet is preferably formed by the vacuum tube formed integrally with the connection element.

According to one preferred embodiment, the connection element comprises a tubular stub for fluidic connection to a vacuum tube in a vacuum-tight manner. This has the advantage that a different vacuum tube can be chosen, depending on the application. For example, vacuum tubes with different tube lengths can be chosen. In such an embodiment, the fluid outlet is preferably formed by the tubular stub.

The object to be achieved is also achieved by a vacuum wound treatment kit which comprises a vacuum wound dressing and a connection unit having the features described above, wherein the distal end section of the connection unit can be fluidically connected to the vacuum wound dressing in a vacuum-tight manner.

In respect of the advantages that can be achieved with the vacuum wound treatment kit, attention is drawn to the relevant statements relating to the connection unit. The features described in connection with the connection unit can be used for the further configuration of the vacuum wound treatment kit.

The object to be achieved is also achieved by a vacuum wound treatment system which comprises a vacuum wound treatment kit having the features described above. The vacuum wound treatment system according to the invention furthermore comprises a vacuum source, wherein the fluid outlet of the connection element can be fluidically connected to the vacuum source in a vacuum-tight manner.

In respect of the advantages that can be achieved with the vacuum wound treatment system, attention is drawn to the relevant statements relating to the connection unit. The features described in connection with the connection unit can be used for the further configuration of the vacuum wound treatment system.

The invention is described below in greater detail by means of the figures, wherein elements that are the same or functionally the same are possibly provided only once with reference signs. The figures serve as an example and should not be interpreted as restrictive. In the figures,

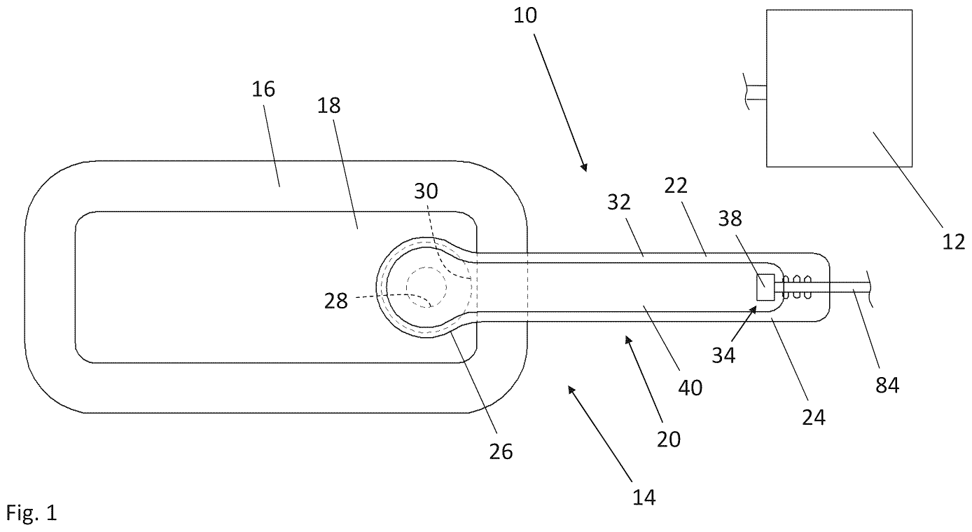

FIG. 1 shows a vacuum wound treatment system comprising a connection unit, which comprises a connection element,

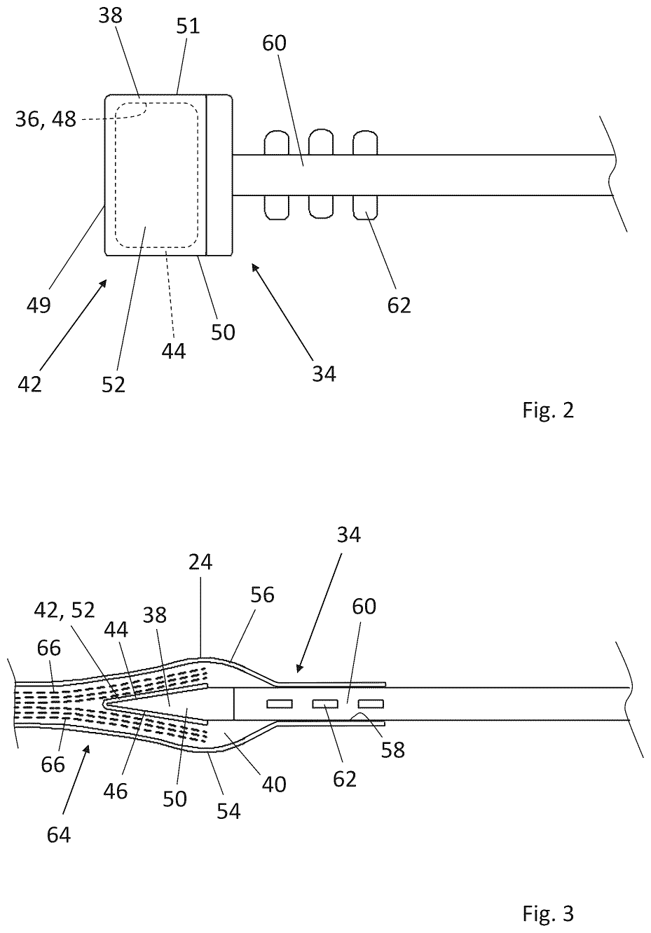

FIG. 2 shows a plan view of the connection element,

FIG. 3 shows a longitudinal section through the connection unit shown in FIG. 1 in the region of the connection element,

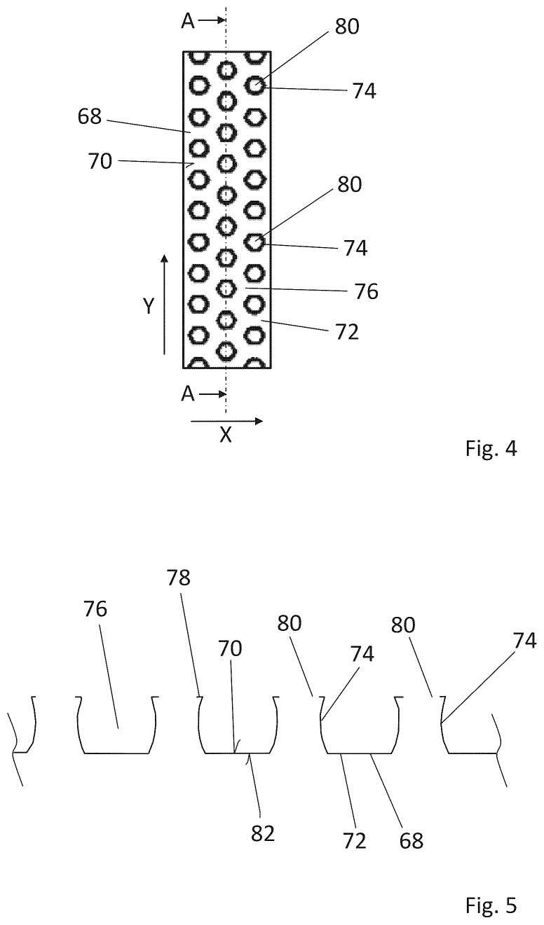

FIG. 4 shows a plan view of a flat material web section, which is arranged as a supporting layer in a suction lumen of the connection unit, and

FIG. 5 shows a section through the flat material web section illustrated in FIG. 4 along section line A-A.

FIG. 1 shows a vacuum wound treatment system 10 for use in the vacuum treatment of wounds. The vacuum wound treatment system 10 comprises a vacuum source 12 and a vacuum wound treatment kit 14. The vacuum wound treatment kit 14 comprises a vacuum wound dressing 16, which is referred to below as a wound dressing 16. The wound dressing 16 comprises an air-impermeable covering layer 18 for airtight sealing of a wound. The vacuum wound treatment kit 14 furthermore comprises a connection unit 20. The connection unit 20 comprises an elongate connecting tube 22 with a proximal end section 24 and a distal end section 26. In the present case, the connecting tube 22 is of single-lumen design.

The distal end section 26 can be fluidically connected in a vacuum-tight manner to a connection opening 28 formed in the covering layer 18. In the vacuum wound treatment system 10 illustrated in FIG. 1, the distal end section 26 is already connected to the connection opening 28, and therefore the connection opening 28 is covered by the distal end section 26. The distal end section 26 comprises an inlet opening 30. The distal end section 26 is arranged on the wound dressing 16 in such a way that the connection opening 28 and the inlet opening 30 of the distal end section 26 are brought into overlap, at least in some region or regions.

The distal end section 26 is widened over an extended area relative to an elongate central section 32 of the connecting tube 22. In the present case, the distal end section 26 is widened in the form of a circular disk. The widening of the distal end section 26 makes it easier to connect the distal end section 26 to the wound dressing 16.

The proximal end section 24 can be fluidically connected to the vacuum source 12 in a vacuum-tight manner. For this purpose, the connection unit 20 comprises a connection element 34 with a fluid inlet 36 and a fluid outlet. The fluid inlet 36 and the fluid outlet are fluidically connected to one another by a fluid duct formed in the connection element 34.

The design of the connection element 34 is explained in greater detail below with additional reference to FIGS. 2 and 3. In this regard, FIG. 2 shows a plan view of the connection element 34. FIG. 3 shows a longitudinal section through the connection unit 20 in the region of the proximal end section 24. Here, the section plane is such that a side view of the connection element 34 is shown.

An inlet section 38 of the connection element 34, said inlet section comprising the fluid inlet 36, projects into a tube lumen 40 of the connecting tube 22 in the region of the proximal end section 24. The fluid inlet 36 is thus arranged in the tube lumen 40. The fluid outlet is arranged outside the tube lumen 40 and can be fluidically connected to the vacuum source 12 in a vacuum-tight manner. Accordingly, the proximal end section 24 of the connecting tube 22 can be fluidically connected to the vacuum source 12 in a vacuum-tight manner by means of the connection element 34.

In the present case, the connection element 34 comprises a vacuum tube 84 formed integrally with the connection element 34. The fluid outlet (not illustrated) is formed by the vacuum tube 84. According to another exemplary embodiment, the connection element 34 comprises, for example, a tubular stub, which forms the fluid outlet of the connection element and can be fluidically connected to a vacuum tube in a vacuum-tight manner.

A filter unit 42 is arranged on the inlet section 38, which filter unit is assigned to the fluid inlet 36. The filter unit 42 is of air-permeable and liquid-impermeable design.

When the vacuum wound treatment system 10 is used as intended, the wound dressing 16 is applied to a wound. The vacuum source 12 is fluidically connected to the wound dressing 16 in a vacuum-tight manner by the connection unit 20. A vacuum can then be produced in the wound space by the vacuum source 12. The vacuum has the effect that wound exudate is drained from the wound and absorbed by the wound dressing 16, e.g. by an absorption layer of the wound dressing 16. Because the filter unit 42 is of air-permeable design, the vacuum can be communicated to the wound space through the filter unit 42. Because the filter unit 42 is of liquid-impermeable design, the filter unit 42 prevents wound exudate from getting into the vacuum source 12. Because the filter unit 42 is arranged on the inlet section 38, the filter unit 42 is clogged by wound exudate only when the absorption capacity of the wound dressing 16 for wound exudate has been exceeded. If the filter unit were arranged on the distal end section 26 and assigned to the inlet opening 30 of the distal end section 26, for example, it would be easier for the filter unit 42 to be clogged even before the absorption capacity of the wound dressing 16 had been exceeded.

The inlet section 38 is designed in such a way as to be widened transversely to the longitudinal extent of the connection element 34. This has the advantage that a fluid inlet 36 with a large opening area can be obtained. Accordingly, it is also possible to use a filter unit 42 with a large filter area, this having the advantage that the filter unit 42 is clogged more slowly by wound exudate.

The inlet section 38 is of wedge-shaped design. For this purpose, the inlet section 38 comprises a first wall section 44 and a second wall section 46, which come together at an acute angle at a front edge 49 of the inlet section 38. In the present case, the acute angle is about 20°. The first wall section 44 and the second wall section 46 are of flat design. As can be seen from FIG. 2, the first wall section 44 is of rectangular design. A corresponding statement also applies to the second wall section 46, which is not visible in FIG. 2.

The fluid inlet 36 comprises an inlet opening 48 formed in the first wall section 44. The inlet opening 48 is of rectangular design. The inlet opening 48 preferably occupies at least 40% of the area of the first wall section 44, particularly preferably at least 60%. An inlet opening 48 is also formed in the second wall section 46. This inlet opening is covered in the figures and is therefore not visible.

The inlet section 38 furthermore comprises a third wall section 50 and a fourth wall section 51. The third wall section 50 and the fourth wall section 51 are extended orthogonally to the first wall section 44 and the second wall section 46. The third wall section 50 and the fourth wall section 51 are of closed design, and therefore wall sections 50 and 51 are free from an inlet opening.

In the exemplary embodiment illustrated in the figures, the filter unit 42 comprises just a single air-permeable and liquid-impermeable membrane filter 52. In the present case, the membrane filter 52 is a PTFE membrane filter 52. The membrane filter 52 is arranged on the inlet section 38 in such a way that it covers the inlet openings 48 in the first wall section 44 and the second wall section 46. For this purpose, the membrane filter 52 is bent around the front edge 49 of the inlet section 38 and rests against the first wall section 44 and the second wall section 46. The membrane filter 52 is preferably materially joined to the inlet section 38, e.g. by a welded joint or by an adhesive joint.

According to another exemplary embodiment, the filter unit 42 has, instead of the single membrane filter 52, a number of membrane filters corresponding to the number of inlet openings 48 for example, wherein each of the membrane filters covers a different one of the inlet openings 48.

The connecting tube 22 comprises a lower or first ply 54 and an upper or second ply 56. The plies 54 and 56 are joined to one another in their edge regions and jointly form the tube wall of the connecting tube 22. In the present case, the plies 54 and 56 are designed as film plies. Accordingly, the connecting tube 22 is a film tube. The plies 54 and 56 are preferably manufactured from polyurethane, polyvinyl chloride, polyethylene, silicone or a mixture thereof. The edge regions of the plies 54 and 56 are preferably joined to one another by an adhesive joint or by a welded joint. The inlet opening 30 of the distal end section 26 is formed in the first ply 54.

The connection element 34 projects through a passage 58 into the tube lumen 40, which passage is formed between the edge region of the first ply 54 and the edge region of the second ply 56. Such an embodiment of the connection unit 20 is easy to implement in terms of manufacturing technology, despite the widened inlet section 38. In the course of manufacture, the connection element 34 is preferably arranged between the two plies 54 and 56. Only then are the two plies 54 and 56 joined to one another in their edge regions. The arrangement of the connection element 34 in the passage 58 formed between the edge regions furthermore has the advantage that a large contact area is created between the outer contour of the connection element 34 and the plies 54 and 56. This makes it easier to pass the connection element 34 through the tube wall of the connecting tube 22 in a fluid-tight manner.

A section 60 of the connection element 34 which is arranged in the passage 58 comprises a plurality of protrusions 62. The protrusions 62 project from the section 60 transversely to the longitudinal extent of the connection element 34 and interact with the edge regions of the plies 54 and 56 to provide positive-locking and clamp-locking retention of the section 60 in the passage 58.

Here, the connection unit 20 furthermore comprises, for example, a supporting unit 64, which supports the connecting tube 22 against collapse, in particular vacuum-induced collapse, and allows fluid to pass through in the longitudinal extent of the suction lumen 40. The supporting unit 64 comprises a plurality of supporting layers 66, which are arranged in such a way as to be stacked one above the other in the suction lumen 40. In the present case, six supporting layers 66 are present. However, there may also be a different number of supporting layers 66.

In the case illustrated here by way of example, the inlet section 38 projects in between two supporting layers 66 arranged immediately adjacent. This avoids a situation where the tube wall of the connecting tube 22 rests flat against the inlet section 38 and thereby closes the fluid inlet 36.

The design of the supporting layers 66 is explained in greater detail below with additional reference to FIGS. 4 and 5. In this regard, FIG. 4 shows a plan view of one of the supporting layers 66. It should be noted here that FIG. 4 illustrates only a segment of the supporting layer 66. Accordingly, the actual outer contour of the supporting layer 66 differs from the outer contour shown in FIG. 4. FIG. 5 shows a section through the supporting layer 66 along the section line A-A shown in FIG. 4.

In the exemplary embodiment illustrated here, the supporting layer 66 is formed by a flat material web section 68. The flat material web section 68 is extended in two surface directions X and Y. In order to support the connecting tube 22, a first side 70 of the flat material web section 68 is formed in a structured manner by raised portions 74 formed integrally with a surface 72 of the flat material web section 68. A continuous interspace 76 is formed between the raised portions 74, thus enabling fluid, such as wound exudate, to be carried through the interspace 76.

In the case of at least some of the raised portions 74, a through opening 80 is formed in the raised ends 78 of the relevant raised portions 74. In the present case, a through opening 80 is formed in the end of each of the raised portions 74. Fluid, e.g. wound exudate, can pass through the flat material web section 68 via the through openings 80 at the ends and can then spread on both sides of the flat material web section 68. The second side 82 of the flat material web section 68, said side facing away from the structured first side 70, is of smooth design.

To form the continuous interspace 76, the raised portions 74 are arranged in such a way as to be isolated from one another in the manner of islands. In the exemplary embodiment illustrated, the shape of the raised portions 74 resembles that of a hyperboloid of one sheet. In the present case, therefore, the raised portions 74 are of hyperboloid design. For this purpose, a lateral wall of the raised portions 74 is curved anticlastically. Alternatively, the raised portions 74 are preferably of conical, i.e. frustoconical, cylindrical or frustopyramidal design.

The raised portions 74 are arranged in a manner distributed in such a way that each of the raised portions 74 is surrounded, at least in a central region of the flat material web section 68, by three to eight further raised portions 74, wherein the further raised portions 74 jointly form a regular polygon. In the present case, the further raised portions 74 jointly form a regular hexagon. At least in the central region of the flat material web section 68, each of the raised portions 74 is therefore surrounded by six other raised portions 74.

In the present case, the flat material web section 68 is a plastic film 68. The plastic film 68 is preferably manufactured from polyethylene, polyurethane, polyvinyl chloride or a mixture thereof. These plastics have the advantage that a flexible connecting tube 22 is obtained, this being associated with high patient comfort. Thus, the flexible embodiment of the connecting tube 22 makes it possible to avoid the occurrence of pressure ulcers, for example. Moreover, the abovementioned plastics also have adequate rigidity. Thus, the plastics ensure that the continuous interspace 76 is maintained during the use of the connection unit 22 despite the vacuum produced.

The raised portions 74 are produced by vacuum deep drawing. For this purpose, an extruded or calendered flat material web, in particular a plastics film web, which is still molten, is preferably passed via a rotary perforated vacuum roller. The flat material web is then drawn into holes in the perforated vacuum roller in some region or regions by a vacuum in the perforated vacuum roller, as a result of which the raised portions 74 are formed. Here, the shape of the raised portions 74 obtained is determined, for example, by the contour of the holes and by the vacuum applied. The vacuum in the perforated vacuum roller is preferably set in such a way that the ends 78 of the raised portions 74 open. The raised portions 74 and the through openings 80 in the ends are thus formed in the same process step.

The embodiment of the supporting layers 66 which has been described above in conjunction with FIGS. 4 and 5 is preferred. However, supporting layers 66 which differ therefrom may also be used. According to another exemplary embodiment, the supporting layers 66 are each formed by a spacing fabric or by an extruded mesh.

1-19. (canceled)

20. A connection unit for vacuum-tight fluidic connection of a vacuum wound dressing to a vacuum source, comprising:
an elongate single- or multi-lumen connecting tube, which comprises a distal end section and a proximal end section, and wherein the distal end section is able to be fluidically connected to the vacuum wound dressing in a vacuum-tight manner; and
a connection element, which comprises a fluid inlet and a fluid outlet, wherein
an inlet section of the connection element, said inlet section comprising the fluid inlet, projects into a tube lumen of the connecting tube in the region of the proximal end section,
wherein the fluid outlet is arranged outside the tube lumen and is able to be fluidically connected to the vacuum source in a vacuum-tight manner, and
wherein a filter unit is arranged on the inlet section, wherein the filter unit is assigned to the fluid inlet and comprises an air-permeable and liquid-impermeable design.

21. The connection unit of claim 20, wherein the filter unit comprises at least one air-permeable and liquid-impermeable membrane filter.

22. The connection unit of claim 21, wherein the fluid inlet comprises a plurality of inlet openings, which are covered by a common membrane filter.

23. The connection unit of claim 20, wherein the inlet section widens transversely to the longitudinal extent of the connection element.

24. The connection unit of claim 20, wherein at least one inlet opening of the fluid inlet is formed in a flat wall section of the inlet section.

25. The connection unit of claim 20, wherein the inlet section comprises a first wall section and a second wall section, which come together at an acute angle at a front edge of the inlet section, and wherein the fluid inlet comprises at least one inlet opening formed in the first wall section and/or at least one inlet opening formed in the second wall section.

26. The connection unit of claim 25, wherein at least one inlet opening is formed both in the first wall section and in the second wall section.

27. The connection unit of claim 25, wherein the inlet section comprises a third wall section and a fourth wall section, which are extended orthogonally to the first wall section and the second wall section, and wherein the third wall section and the fourth wall section are closed.

28. The connection unit of claim 20, wherein the inlet section is wedge-shaped.

29. The connection unit of claim 20, wherein the connecting tube comprises a first ply and a second ply, wherein the first ply and the second ply each comprise an edge region, and wherein the first ply and the second ply are materially joined to one another at the edge regions and jointly surround the tube lumen.

30. The connection unit of claim 29, wherein the connection element projects through a passage into the tube lumen, wherein the passage is formed between the edge region of the first ply and the edge region of the second ply.

31. The connection unit of claim 30, wherein a section of the connection element which is arranged in the passage comprises at least one protrusion for positive-locking and/or clamp-locking retention of the section in the passage.

32. The connection unit of claim 20, wherein a supporting unit is arranged in the tube lumen, said unit supporting the connecting tube against collapse and allowing fluid to pass through in the longitudinal extent of the tube lumen, wherein the supporting unit comprises at least one supporting layer, and wherein the connection element and the supporting layer overlap in some region or regions.

33. The connection unit of claim 32, wherein the supporting layer is formed by a flat material web section, which, in order to support the connecting tube, is formed in a structured manner by raised portions formed integrally with a surface of the flat material web section, wherein a continuous interspace, which allows fluid to pass through in the longitudinal extent of the tube lumen, is formed between the raised portions.

34. The connection unit of claim 32, wherein the supporting unit comprises at least two supporting layers arranged stacked one on top of the other, and in that the connection element projects between the supporting layers.

35. The connection unit of claim 20, wherein the connection element comprises a vacuum tube, which is formed integrally with the connection element and is able to be fluidically connected to the vacuum source in a vacuum-tight manner.

36. The connection unit of claim 20, wherein the connection element comprises a tubular stub for fluidic connection to a vacuum tube in a vacuum-tight manner.

37. A vacuum wound treatment kit comprising a vacuum wound dressing and the connection unit of claim 20, wherein the distal end section of the connecting tube is able to be fluidically connected to the vacuum wound dressing in a vacuum-tight manner.

38. A vacuum wound treatment system comprising a vacuum wound treatment kit of claim 37, and comprising a vacuum source, wherein the fluid outlet of the connection element is able to be fluidically connected to the vacuum source in a vacuum-tight manner.
